(19) United States
(12) Patent Application Publication
(10) Pub. No.: US 2014/0052183 A1
(43) Pub. Date: Feb. 20, 2014

(54) Posterior Spine Attachment Device for Hardware and Paraspinal Musculature

(71) Applicant: FreeseTEC Corporation, Bryn Mawr, PA (US)

(72) Inventor: Andrew Freese, Chester Springs, PA (US)

(73) Assignee: FreeseTEC Corporation, Bryn Mawr, PA (US)

(21) Appl. No.: 13/961,355

(22) Filed: Aug. 7, 2013

Related U.S. Application Data
(60) Provisional application No. 61/682,039, filed on Aug. 10, 2012.

(51) Int. Cl. A61B 17/04; A61B 17/70

(52) U.S. Cl. CPC A61B 17/0401; A61B 17/7062; A61B 17/7002
USPC 606/248; 606/279

(57) ABSTRACT

Devices, kits, and methods for stabilizing the spine and replacing spinous processes removed during spine surgery are provided. The device has a suitable configuration to attach to both spine surgery hardware and to the paraspinal muscles and fascia. The device contains a muscle attachment portion, one or more connectors, and one or more cross connectors. Each cross connector contains a pair of connection portions configured to attach to hardware that is implanted in the spine, such as screw heads or rods. The muscle attachment portion contains a plurality of openings for the attachment of the paraspinal muscles and fascia. Following spine surgery, a surgeon attaches the device to the hardware implanted in the surgical site, and sutures the paraspinal muscles to the openings. Thus the device provides a direct attachment to the paraspinal musculature and fascia, and thereby stabilizes the spine.

### CROSS-REFERENCE TO RELATED APPLICATION

This application claims priority to U.S. Provisional application Ser. No. 61/682,039, filed on Aug. 10, 2102. The disclosure of U.S. Ser. No. 61/682,039 is herein incorporated by reference.

### FIELD OF THE INVENTION

This invention relates to devices and methods used in spine surgery, particularly devices and methods used to attach spinal vertebrae to the paraspinal musculature after spine surgery.

### BACKGROUND OF THE INVENTION

One of the most common surgical procedures in the spine involves a decompression or laminectomy, which removes the muscular attachments of the paraspinal musculature to the spinous process and lamina (the posterior bony elements of the spine), and then the posterior bony elements of the spine, particularly the spinous processes and lamina. See FIG. 1. The lamina is a posterior arch of the vertebral bone lying between the spinous process, which juts out in the midline, and the more lateral pedicles and transverse processes of each vertebra. The pair of laminae, along with the spinous process, make up the posterior wall of the bony spinal canal. Often additional bony or soft tissue removal involves disrupting additional structural elements of the spine, including the facet joints and/or the discs. Current attempts to recreate the stability of the spinal column following such procedures include the placement of pedicle screws or lateral mass screws connected by a rod construct, and at times a cross connector. A flaw in this process is that the attachments of the paraspinal musculature to the spine are never reconstituted, leaving the spine vulnerable, less stable, and more likely to develop cosmetic deformity.

Upon closure of the wound at the end of spine surgery, the muscle layer is commonly closed with individual sutures that incorporate the muscle and fascia and the two split sides are sewn to each other. This closure technique can lead to muscular atrophy, involution of the overlying skin, cosmetic defects, and the loss of the integrity of the spine-ligament-muscular complex that provides integral stability to the spine. Instability of the spine has been documented to contribute, and in many cases, cause pain, deformity and dysfunction. Therefore, prior to undertaking surgery in the spine, virtually all patients are encouraged to undergo aggressive physical therapy with the goal of strengthening the muscular support and its attachments to the spine, and hopefully reducing pain and averting surgery in the first place.

There is a need for improved surgical devices and methods for spinal surgery which prevent or reduce the severity of deformity, dysfunction and/or pain following spine surgery.

Therefore it is an object of the invention to provide devices and kits for use in surgical procedures in the spine that improve the stability of the spinal column and/or prevent the development of cosmetic deformity following such procedures.

It is a further object of the invention to provide improved methods for increasing the stability of the spine following surgical procedures.

### SUMMARY OF THE INVENTION

Devices, kits, and methods for stabilizing the spine and effectively replacing spinous processes that are removed during spine surgery are provided. The device has a suitable configuration to attach to hardware that is inserted into the patient's spine during surgery and to the paraspinal muscles and fascia. The device contains at least one muscle attachment portion and one or more connectors, in a preferred embodiment, the device also contains one or more cross connectors. Each connector is configured to attach to a cross connector. Each cross connector contains a pair of connection portions configured to attach to hardware that is implanted in the spine, such as screw heads or rods. The muscle attachment portion, also referred to as the fin portion, contains one or more muscle attachment areas that are suitable for the attachment, such as via suturing, of the paraspinal muscles and fascia. In one embodiment, the muscle attachment area contains a material that allows for suturing, such as a biocompatible textile (e.g. a synthetic polyester fabric, preferably formed from polyethylene terephthalate fibers, such as Dacron®), or a mesh material. In another embodiment, the muscle attachment area is a plurality of openings, such as plurality of holes. Optionally, the muscle attachment portion further comprises one or more drugs that are released following implantation over a period of time ranging from one week to five months, preferably ranging from one week to eight weeks.

Following spine surgery including the step of removing the spinous process in one or more vertebra(e), a surgeon can attach the device to the hardware implanted in the surgical site, and then attach the paraspinal muscles and fascia to the muscle attachment area of the muscle attachment portion, typically via sutures. Thus the device provides a direct attachment to the paraspinal musculature and fascia, and thereby further strengthens and stabilizes the spine. Use of the device also prevents or reduces the formation of cosmetic deformities as the spinal wound heals and prevents or reduces involution of the tissues following healing, which typically create a crater or valley effect in a patient's back following spine surgery. Attachment of the paraspinal muscles and fascia to the device also reduces the likelihood that the paraspinal musculature will attach to dura mater, the spinal cord and/or nerves following the surgery, and reduces the formation of scar tissue and adhesions.

### BRIEF DESCRIPTION OF THE DRAWINGS

FIG. 1 is illustration of an upper view of vertebrae, showing the lamina and spinous process

FIGS. 2A-E are different views of an exemplary fixation device attached via two fixation rods to a plurality of pedicle screws that can be secured to adjacent vertebrae. The fixation device contains a rigid muscle attachment portion with a plurality of holes and three connectors, which attach to three cross connectors. The cross connectors are configured to attach to each of the fixation rods. FIG. 2A is an anterior-posterior view of the system; FIG. 2B is a sagittal view; FIG. 2C is an isometric view; FIG. 2D is an axial view; FIG. 2E is an enlarged portion of FIG. 2D, providing a detailed view of the muscle attachment portion and the connector portion.

FIGS. 3A-3D are illustrations of rigid muscle attachment portions, also referred to herein as the “fin portion”, with a plurality of holes throughout the muscle attachment portion. FIGS. 3A and 3B are perspective and plan views, respectively, of a small fin portion. FIGS. 3C and 3D are plan views of medium and large size, respectively, fin portions.

FIGS. 4A-4D are illustrations of mesh muscle attachment portions, also referred to herein as the “fin portion”, with one or more mesh areas in the device. FIGS. 4A and 4B are perspective and plan views, respectively, of a small fin portion. FIGS. 4C and 4D are plan views of medium and large size, respectively, fin portions.

FIGS. 5A and 5B are different views of a fixation device attached to two fixation rods which are attached to multiple pedicle screws. The fixation device contains a segmented rigid muscle attachment portion, containing three fin portions with a plurality of holes, and three connectors, which attach to three cross connectors. The cross connectors are configured to attach to each of the fixation rods. FIG. 5A is a sagittal view and FIG. 5B is an isometric view.

FIGS. 6A-E are different views of a fixation device attached directly to the screw heads of the pedicle screws. The fixation device contains a rigid muscle attachment portion with a plurality of holes and three connectors, shown attached to three cross connectors. FIG. 6A is an anterior-posterior view of the system; FIG. 6B is a sagittal view; FIG. 6C is an isometric view; FIG. 6D is an axial view; FIG. 6E is an enlarged portion of FIG. 6D, providing a detailed view of the muscle attachment portion and the cross connectors.

FIG. 7 is an isometric view of a fixation device with single muscle attachment portion containing multiple mesh areas. Like the device illustrated in FIGS. 6A-E, this device attaches directly to the screw heads of the pedicle screws.

FIG. 8 is an isometric view of a fixation device attached via two fixation rods to a plurality of pedicle screws. The fixation device contains a segmented rigid muscle attachment portion, containing three fin portions, each of which contains a mesh area and three pairs of connectors, which attach to three cross connectors. The cross connectors attach directly to the screw heads of the pedicle screws.

FIG. 9A is an isometric view of a fixation device attached via two fixation rods to a plurality of pedicle screws. The device contains a segmented rigid muscle attachment portion, containing three fin portions, each of which contains a mesh area and three connectors, which attach to three cross connectors. The connectors are in the form of a clamp that is integral with each rigid muscle attachment portion. The cross connectors attach directly to the screw heads of the pedicle screws. FIGS. 9B and 9C are orthographic views of the fixation device depicted in FIG. 9A in the absence of the rods and pedicle screws. FIGS. 9B is a top view; and FIG. 9C is a side view. FIG. 9D is a side view of the fixation device depicted in FIG. 9A.

FIG. 10 is a lateral view of a fixation device that contains a rigid muscle attachment portion with a plurality of holes and two connectors, shown attached to two cross connectors. The cross connectors are configured to attach to each of the fixation rods.

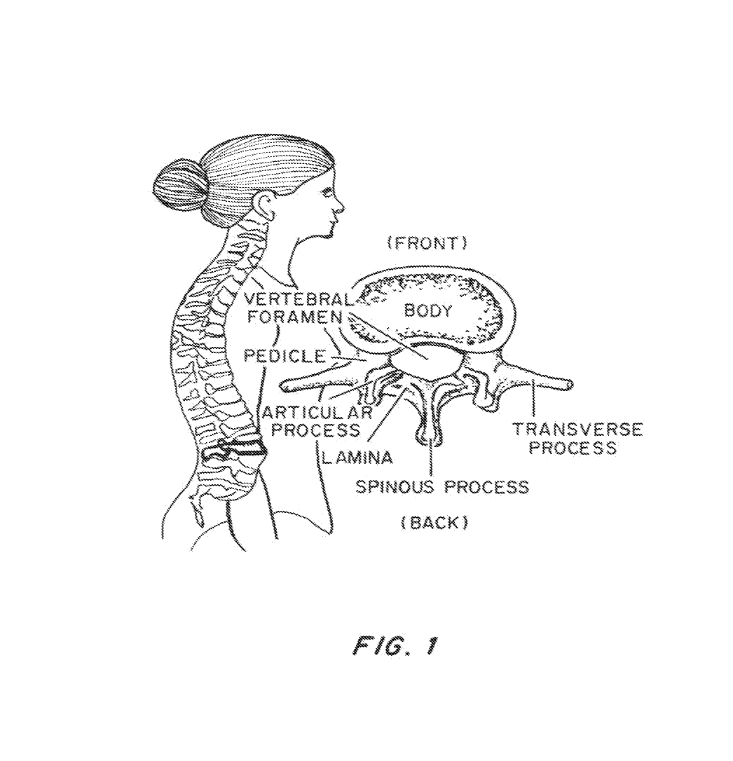

### DETAILED DESCRIPTION OF THE INVENTION

### I. Devices for Attachment of the Paraspinal Muscles and Fascia

A device for replacement of spinous processes that are removed during spine surgery has been developed. The device may be used in any area of the spine, i.e. the cervical, thoracic and/or lumbar portions of the spine.

The device has a suitable configuration to attach to hardware that is inserted into the patient's spine during surgery and to the paraspinal muscles and fascia. Exemplary fixation devices (10) are illustrated in the figures. In a preferred embodiment, the fixation device (10) contains a muscle attachment portion (20), herein referred to as the “fin portion”, and one or more connectors (40), which attach to one or more cross connectors (50). The cross connectors are configured to attach to each of the fixation rods (80a and 80b) or directly to the screw heads (90a, b, and c) of the pedicle screws (98a, b, and c). When fully assembled, the fixation device, rods and screws form a spinal fixation system (100, 200, 300, 400, 500, or 600), where the muscle attachment portion is superior to the cross connectors and positioned at approximately a 90° angle relative to the upper portion of the connectors.

A. Muscle Attachment Portion

The muscle attachment portion (20) can have any suitable shape that provides attachment areas to which the paraspinal muscles and fascia can connect and is sufficiently long to be placed in the portion of the patient's spine from which the spinous processes were removed from the vertebrae. In one embodiment the muscle attachment portion is a single unified portion. Preferably in this embodiment, the lateral cross section of the muscle attachment portion has a shape comprising at least two long sides having the same length, wherein the long sides are longer than each of the other sides. In another embodiment the muscle attachment portion is formed from two or more smaller segments, which are aligned in the same plane when assembled.

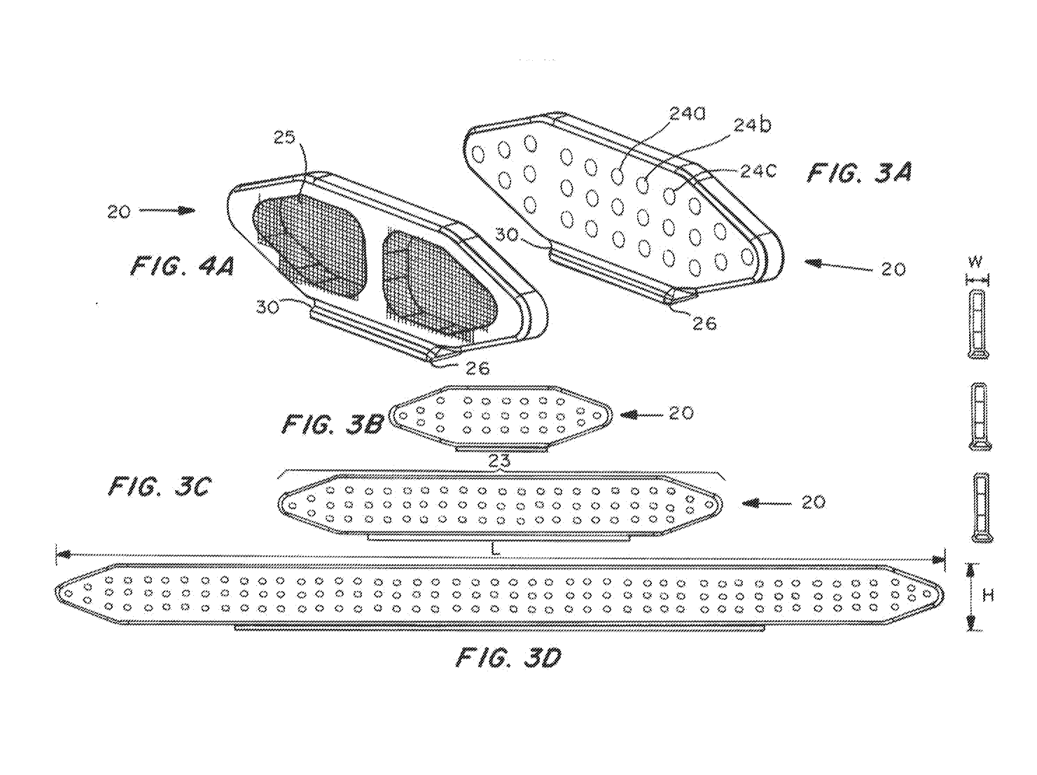

Suitable shapes are illustrated in FIGS. 3A-D, 4A-D and 9D; however additional shapes that meet these functions are also suitable. For small segments, the length (l) of each segment of the muscle attachment portion typically is at least about 1 cm and be up to about 5 cm in length, preferably up to about 3 cm in length. The total length for the muscle attachment portion, i.e. all of the segments or length for a single unified muscle attachment portion, is typically at least 2 cm and can be as long as about 30 cm. The longer muscle attachment portions, such as with lengths of greater than 20 cm, preferably less than or equal to about 30 cm, more preferably less than or equal to about 25 cm. For other surgical treatments, typical lengths (l) for the muscle attachment portion range from 20 mm to 300 mm, preferably from 40 mm to 100 mm, more preferably 80 mm. Typical heights (h) for the muscle attachment portion range from approximately 1 mm to approximately 3 cm, preferably from approximately 2 mm to approximately 1.5 cm. Typical thickness, i.e. widths (w), for the muscle attachment portion range from 1 mm to 5 mm, preferably from 1.5 mm to 3 mm, more preferably 2 mm.

a. Muscle Attachment Areas

The muscle attachment portion contains one or more muscle attachment areas (23) that are suitable for the attachment, such as via suturing, of the paraspinal muscles and fascia. The one or more muscle attachment areas (23) allow a surgeon to attach the muscle and/or fascia at multiple points along the muscle attachment portion, thereby reducing the stretch on the muscle and/or fascia and reducing the likelihood of pullout or tearing off of the fin portion. In one embodiment, the muscle attachment area contains a material (25) that allows for suturing, such as a biocompatible textile (e.g. a synthetic polyester fabric, preferably formed from polyethylene terephthalate fibers, such as Dacron®), or a biocompatible mesh material. In another embodiment, the muscle attachment area is a plurality of openings (24a, b, c), such as plurality of holes in the muscle attachment portion.

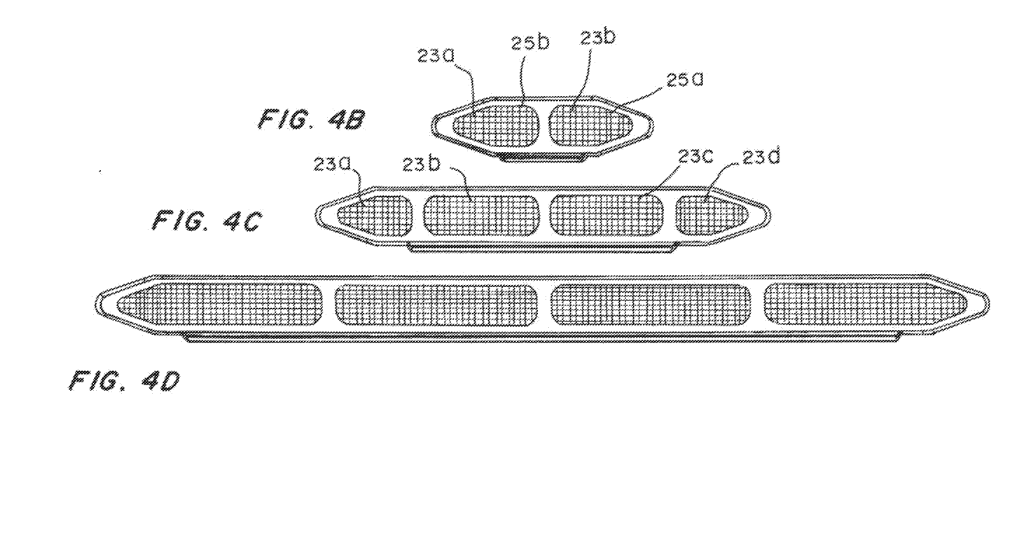

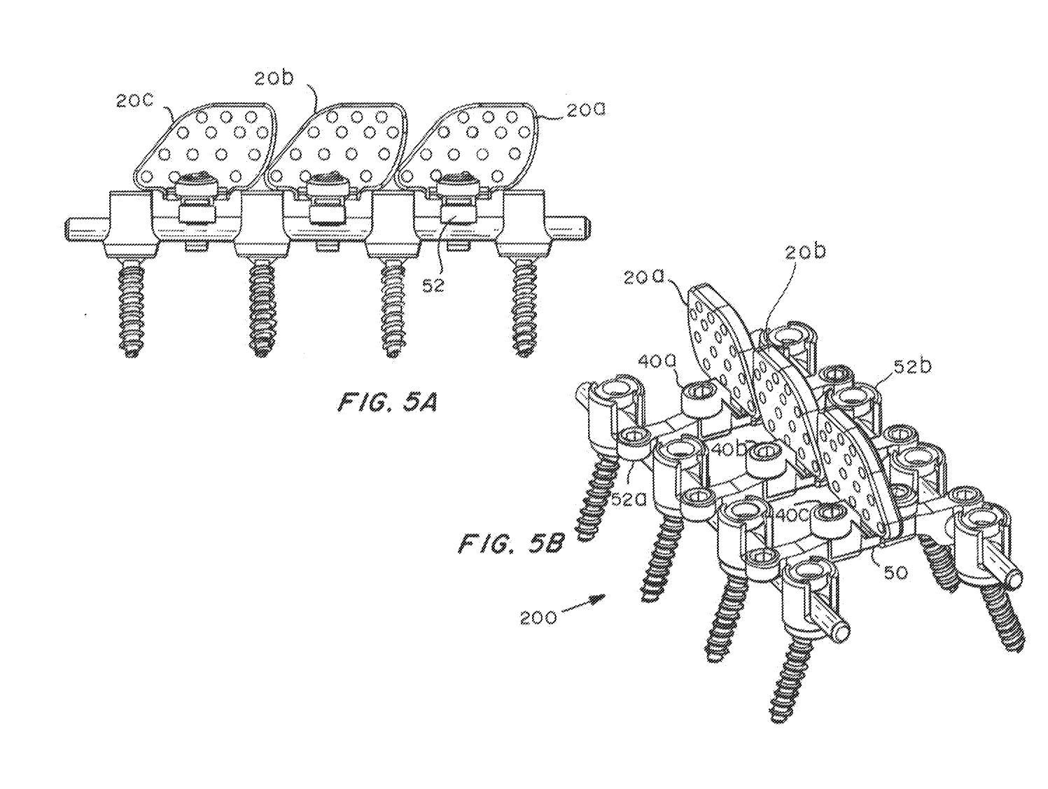

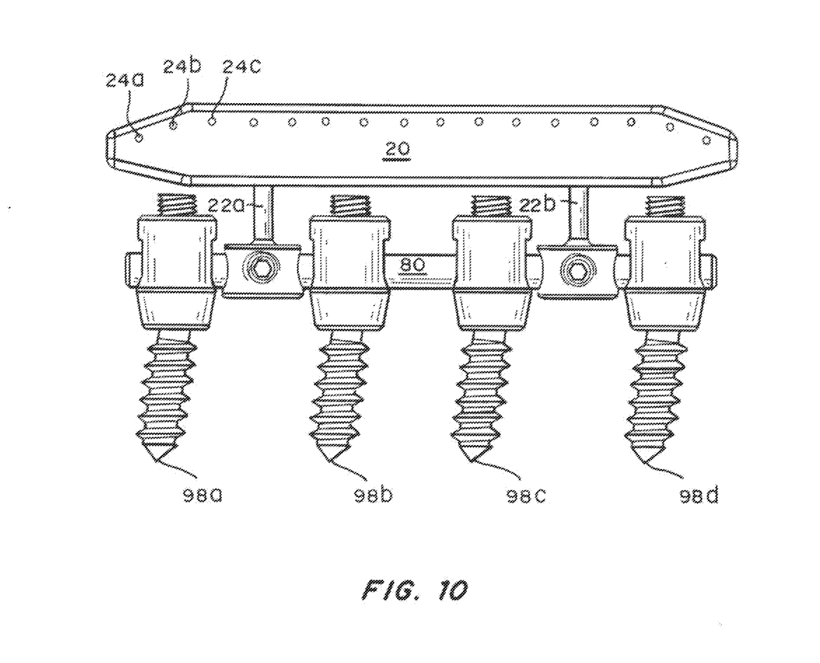

As shown in FIGS. 3A-D, the muscle attachment area may be a plurality of holes (24a, b, c) located throughout the length of the muscle attachment portion or along the length of the upper region of the muscle attachment portion (see FIG. 10). Typically the holes are spaced equally, and typically have the same size, although different sizes and spacings may be used. Alternatively, as shown in FIGS. 4A-D, the muscle attachment area contains a material that allows for suturing (25a, and 25b), such as a mesh.

In use, a surgeon typically sutures the paraspinal musculature and/or fascia to the muscle attachment area.

In one embodiment, the muscle attachment portion is formed from a porous material or a material with striations suitable to encourage muscular attachments to the device, thereby further strengthening the patient's spine.

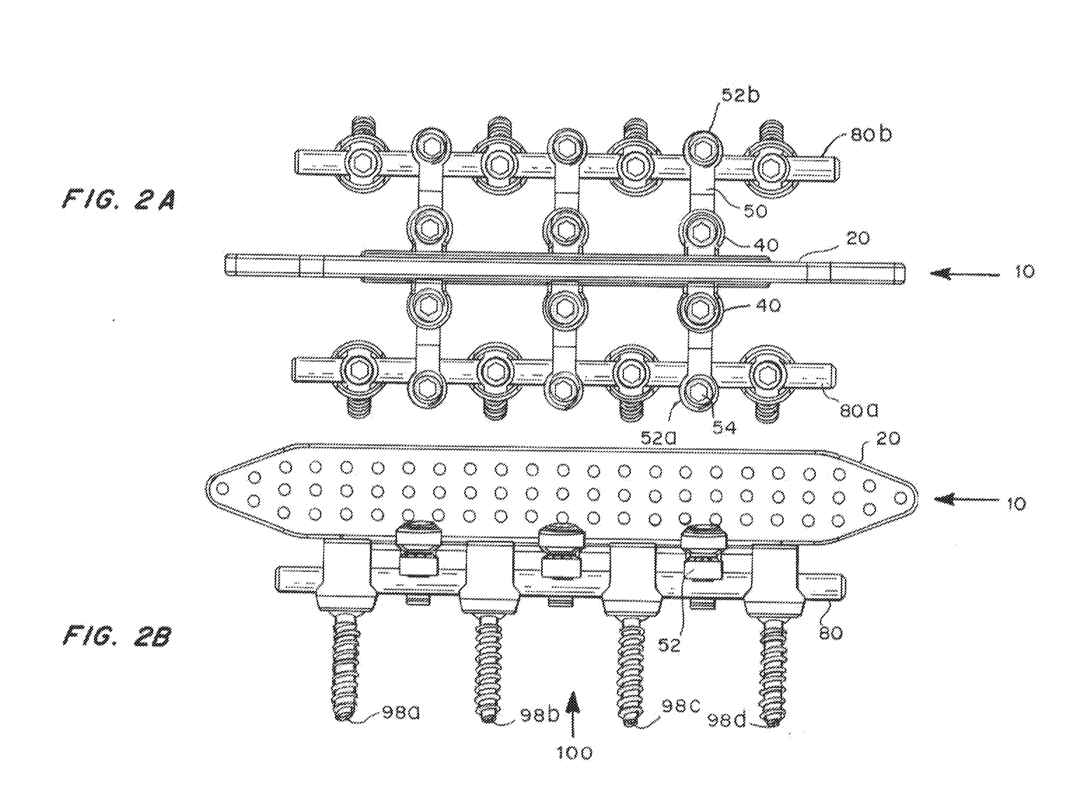

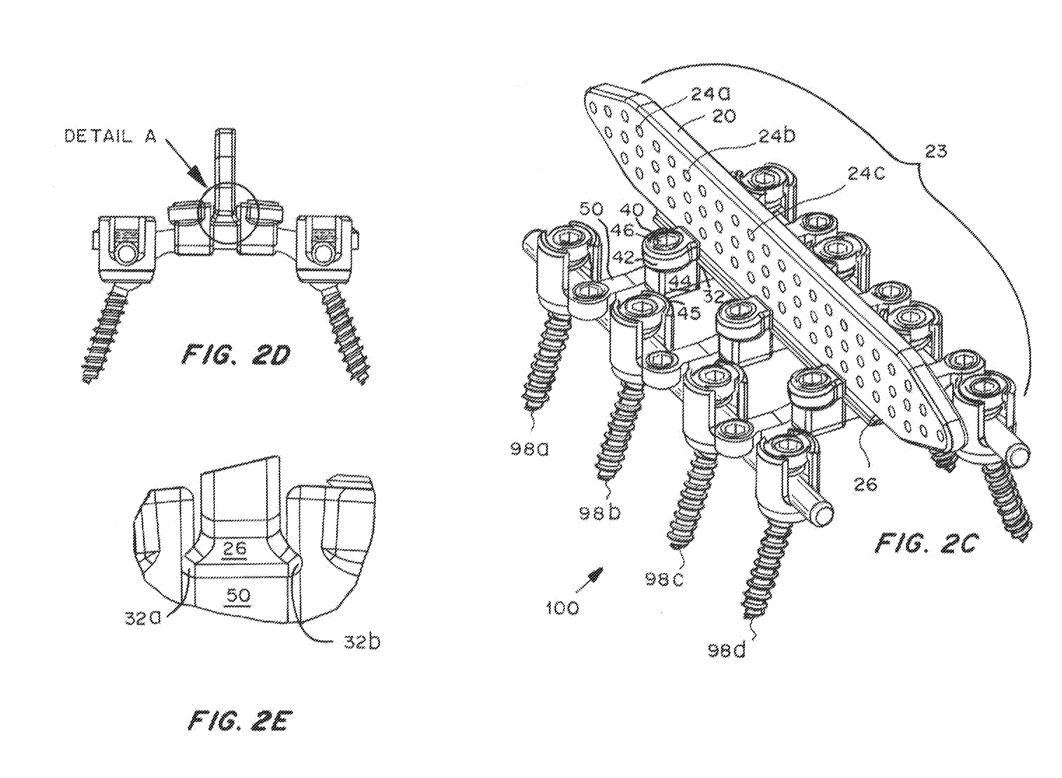

b. Flange In some embodiments, the muscle attachment portion contains a flange (26) at its lower region (30) (see, e.g. FIGS. 3A-D, 4A-D), which fits in a groove (32a and 32b) in the adjacent side of each connector. When fully assembled, the flange is superior to the cross-connector (50) (see, e.g. FIG. 2E).

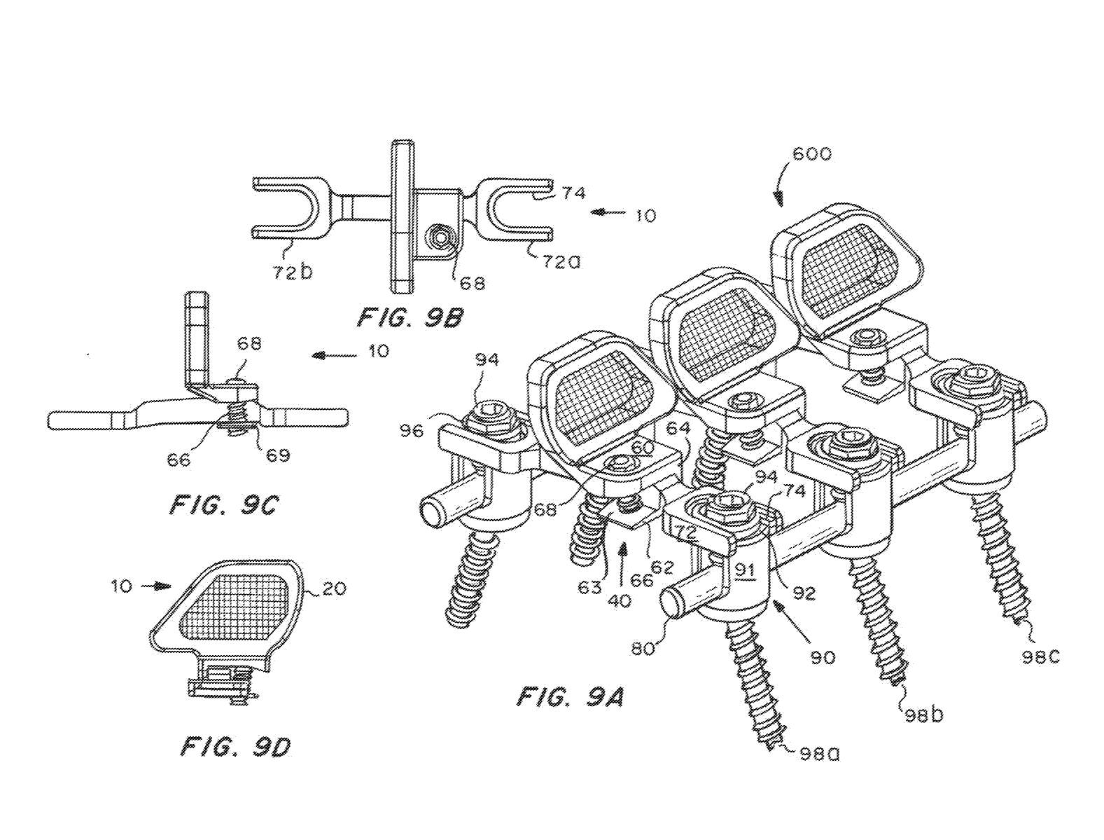

In another embodiment, in place of a flange, a rod (see, e.g. FIG. 10) or other suitable attachment element connects the muscle attachment portion to each connector. These attachment elements typically have a length ranging from 10 mm to 50 mm, preferably from 20 mm to 35 mm, more preferably 30 mm, and a diameter ranging from 3 mm to 6.5 mm, preferably from 3.5 mm to 5.5 mm, more preferably 4.0 mm. As shown in FIG. 9, the proximal end (21) of the rod attaches to the muscle attachment portion. The distal end (23) of the rod (22) contains a suitable shape for attachment to the connector(s) (40) or directly to the cross connector(s) (50).

1. Materials

The muscle attachment portion is formed from any biocompatible material, including biodegradable and non-biodegradable materials. As used herein, “biocompatible materials” are those which do not elicit an acute inflammatory response when implanted into the muscle of an animal such as a mouse. Preferred non-biodegradable polymers include polyesters, polycarbonates, polyethylene, polyamides, and nylon. Ceramics and natural bone materials such as hydroxyapatite can also be used. In one embodiment, the muscle attachment portion is formed from a biodegradable material. Preferably the biodegradable materials degrade over a period of time ranging from two months to three years following implantation in a patient's spine. Examples of biodegradable polymers include polyesters such as poly(lactide-co-glycolide), polyanhydrides, and polyhydroxyalkanoates.

Biodegradable polymers for medical uses must degrade into non-toxic metabolites. Medical devices must also be nonpyrogenic, i.e., the products must not produce fever reactions when administered to patients. The presence of bacterial endotoxin (which is an integral component of the outer cell surface of Gram-negative bacteria), in the product is by far the largest concern of manufacturers in achieving nonpyrogenation. (Weary and Pearson, BioPharm., 1:22-29 (1988)). The U.S. Food and Drug Administration (FDA), for example, requires the endotoxin content of medical devices be less than 20 U.S. Pharmacopeia (USP) endotoxin fluid, where the content must not exceed 2.15 USP endotoxin units per device. U.S. Pat. No. 7,906,135 to Williams, et al. discloses polyhydroxy alkanoates (PHAs) from which pyrogen has been removed for use in numerous biomedical applications, including medical devices.

In one embodiment, the muscle attachment portion is formed from synthetic polymers. Synthetic polymers produce materials that are biocompatible and are not contaminated by biological materials. Additionally, synthetic polymers generally have more reproducible synthesis and degradation both in vitro and in vivo. Synthetic polymers may be modified to produce materials with different properties (e.g. by changing molecular weight and/or functional groups).

Representative synthetic polymers include but are not limited to poly(hydroxy acids) such as poly(lactic acid), poly(glycolic acid), and poly(lactic acid-co-glycolic acid), polyglycolides, polylactides, poly(lactide-co-glycolide) copolymers and blends, polyanhydrides, polyorthoesters, polyamides, polycarbonates, polyalkylenes such as polyethylene and polypropylene, polyalkylene glycols such as poly(ethylene glycol) (PEG), polyalkylene oxides such as poly(ethylene oxide) polyvinyl alcohols, poly(valeric acid), and poly(lactide-co-caprolactone), derivatives, copolymers and blends thereof. As used herein, “derivatives” include polymers having substitutions, additions of chemical groups, for example, alkyl, alkylene, hydroxylations, oxidations, and other modifications routinely made by those skilled in the art.

Examples of preferred biodegradable polymers include polymers of hydroxy acids such as lactic acid and glycolic acid, polylactide, polyglycolide, poly(lactide-co-glycolide), and copolymers with PEG, PHAs, polyanhydrides, poly(ortho)esters, polyurethanes, poly(butyric acid), poly(valeric acid), poly(lactide-co-caprolactone), blends and copolymers thereof.

In one embodiment, the muscle attachment portion is formed from a biocompatible, non-biodegradable material, such as polyaryletherketones (PAEKs), preferably poly(aryl-ether-ether-ketone) (PEEK), titanium or stainless steel. These materials are typically used in the hardware that is inserted into the spine. Titanium is strong, lightweight, weighing 56% as much as steel, and it is one of the few materials that bone grows into and on. However, titanium, like all metals, has the drawback in that it is not translucent to X-rays or MRI scans. So once installed, it can blur or hide anatomical changes. But unlike steel, titanium is non-ferrous, so magnets used in MRI machines will not exert a force on them. have been increasingly employed as biomaterials for orthopedic, trauma, and spinal implants.

PAEK is a family of high temperature thermoplastic polymers, consisting of an aromatic backbone molecular chain, interconnected by ketone and ether functional groups. These polymers are strong, inert, and biocompatible. Due to its strength and relative inertness, PEEK is broadly accepted as a radiolucent alternative to metallic biomaterials in the spine community. Preferably the muscle attachment portion is formed from PEEK.

c. Therapeutic or Diagnostic Agents

Optionally, the muscle attachment portion contains one or more therapeutic and/or diagnostic agents, which are released from the device following its implantation in the patient. A variety of agents can be incorporated into and released from the muscle attachment portion. Suitable agents include, but are not limited to, analgesics, anesthetics, antibiotics, steroids, antibodies against vascular endothelial growth factor (VEGF), such as bevacizumab. The agents may be released in an effective amount to reduce pain, such as for a period of time ranging from two to eight weeks following surgery. For agents that inhibit or reduce scar formation, the agent may be released in an effective amount for a time period, such as ranging from two to eight weeks following surgery.

Drugs for use in the devices include the following categories and examples of drugs and alternative forms of these drugs such as alternative salt forms, free acid forms, free base forms, and hydrates: analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate); antibiotics (e.g., vancomycin, neomycin, streptomycin, chloramphenicol, cephalosporin, ampicillin, penicillin, tetracycline, and ciprofloxacin); and anti-inflammatories (e.g., non-steroidal such as indomethacin, ketoprofen, flurbiprofen, naproxen, ibuprofen, ramifenazone, and piroxicam, steroidal anti-inflammatories such as cortisone, dexamethasone, fluazacort, celecoxib, rofecoxib, hydrocortisone, prednisolone, and prednisone.

B. Connectors

At least one connector (40) attaches the lower region (30) of the muscle attachment portion (20) to the one or more cross-connectors (50).

In some embodiments, such as illustrated in FIGS. 2A-E, 5A, 5B, 6A-E, 7, and 8, the device contains at least two connectors (40), with one located on each side of the muscle attachment portion. The two connectors form a pair of connectors, which serve as clamps to maintain the muscle attachment portion in its desired position. When clamped in place, the muscle attachment portion is positioned at an approximately 90° angle relative to the upper portion of the connector(s) and/or the cross connector.

Each connector (40) includes an upper portion (42) and a lower portion (44). The upper portion (42) contains a threaded shaft in its center into which a screw or a nut (46) fits. The lower portion (44) contains a slot (45) which has a suitable size and shape to fit around and in slidable relation with the cross-connector (50). The side of the lower portion (44) proximal to the muscle attachment portion (20) contains a groove (32) which has a suitable size and shape to receive the flange (26) of the muscle attachment portion. The screw/nut(s) are tightened to attach the muscle attachment portion to the one or more cross connectors.

The device may contain more than one pair of connectors, and preferably contains at least two pairs of connectors, more preferably at least three pairs of connectors. The number of pairs of connectors depends on the size (e.g. length) of the muscle attachment portion; the longer the muscle attachment portion, the greater the number of connectors.

In another embodiment, such as illustrated in FIGS. 9A-E, the connector (40) is in the form of a clamp with an upper portion (60), a lower portion (62) and a side portion (64), the opposite side forms an opening (63). The opening has a suitable size and shape to allow the cross connector (50) to fit inside the opening, such that the cross connector is in slidable relation to the inner surface of each of the upper, lower and side portions. The upper portion (60) contains a first threaded bore and the lower portion (62) contains a second threaded bore, which is coaxial with the first threaded bore so that a single screw can pass through both holes. A screw (66) fits through the first threaded bore in the upper portion and through the second threaded bore in the lower portion. A nut or other locking element (69) is located on the posterior surface of the lower portion so that the screw and nut can be tightened, thereby attaching the muscle attachment portion (20) to the cross connector (50) via the connector (40).

As shown in FIGS. 9A-E, the connector (40) is integral with the muscle attachment portion. In this embodiment, a flange is not required to attach the muscle attachment portion to the connector.

The connectors are formed from any suitable biocompatible material, and may be formed from the same material or a different material than the muscle attachment portion. Preferably the connector is formed from titanium or stainless steel.

C. Cross Connectors

The fixation device can attach to standard hardware used in spinal surgery, including, but not limited to, pedicle screws, lateral mass screws, and rod constructs using one or more cross connectors. Preferably the cross connectors are formed from titanium or stainless steel.

Cross connectors are placed in a generally transverse direction relative to the spinal rods and link adjacent spinal rods across the spinal midline to provide a rigid and stable construct. Any suitable cross connector can be used, including both fixed and adjustable, or multi-axial, cross connectors. Examples of suitable cross connectors include, but are not limited to, the EXPEDIUM™ SFX™ Cross Connector System by DePuy Spine™, the Spinal Fixation System (SFS) by Orthofix® (typically intended for non-cervical use). Exemplary cross connectors, particularly their systems for attachment to screw heads or rods, are also described in the literature, such as in U.S. Pat. No. 7,645,294 to Kalfas et al. and U.S. Pat. No. 7,628,799 to Richelsoph et al., the disclosures of which are incorporated herein by reference.

FIGS. 2A-E, 5A, and 5B illustrate exemplary fixation devices that attach to one or more cross connectors, where the cross connectors contain a pair of connection portions (52a and 52b) that attach to the fixation rods (80a and 80 b). The connection portions contain a C-shaped channel having a suitable size and shape to accept the rod. The anterior portion of the connection portion contains a screw hole, preferably a threaded screw hole, which has a suitable size to permit a screw (54) to pass through and contact the rod. As the screw is turned, it pushes the rod against the C-shaped channel until it securely connects the connection portion with the rod.

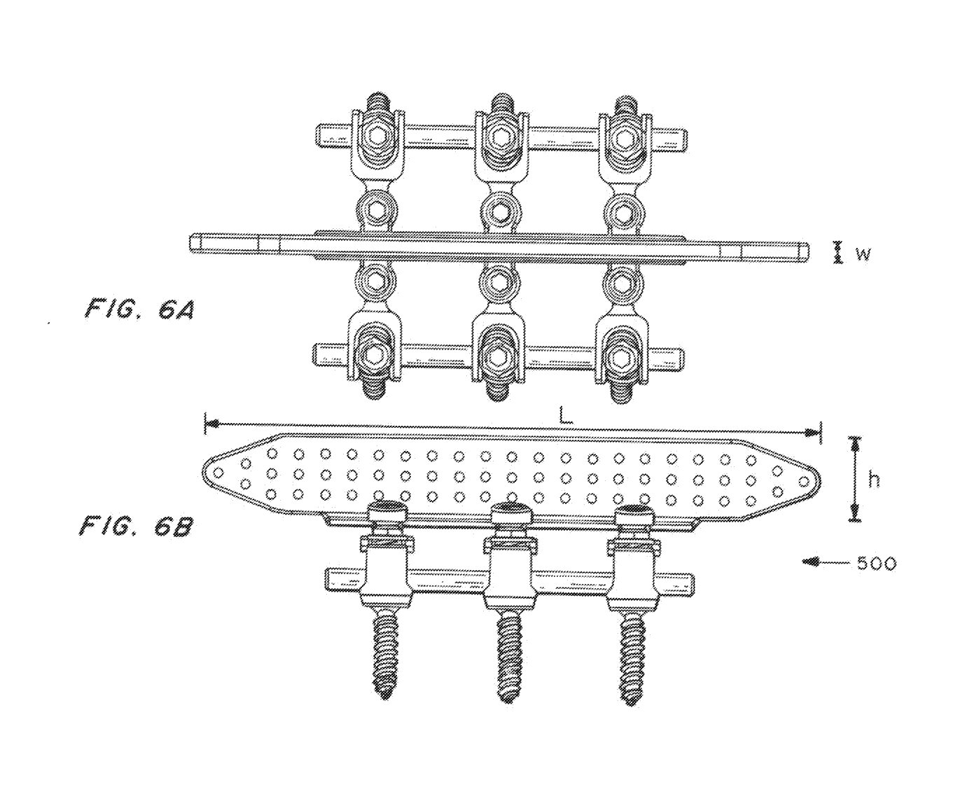

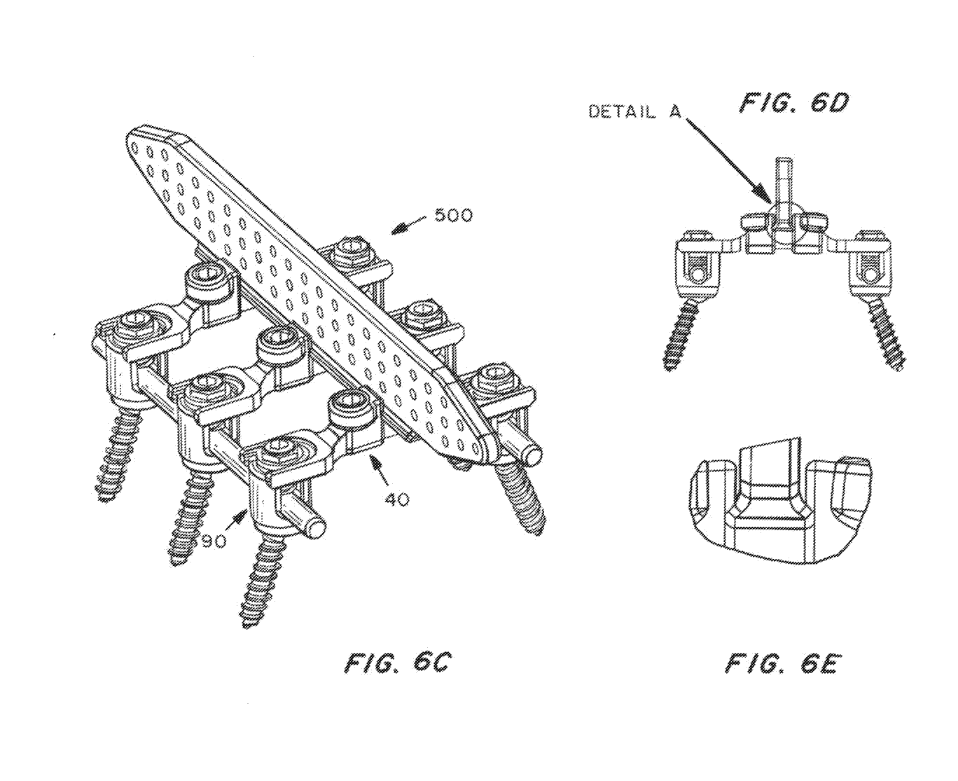

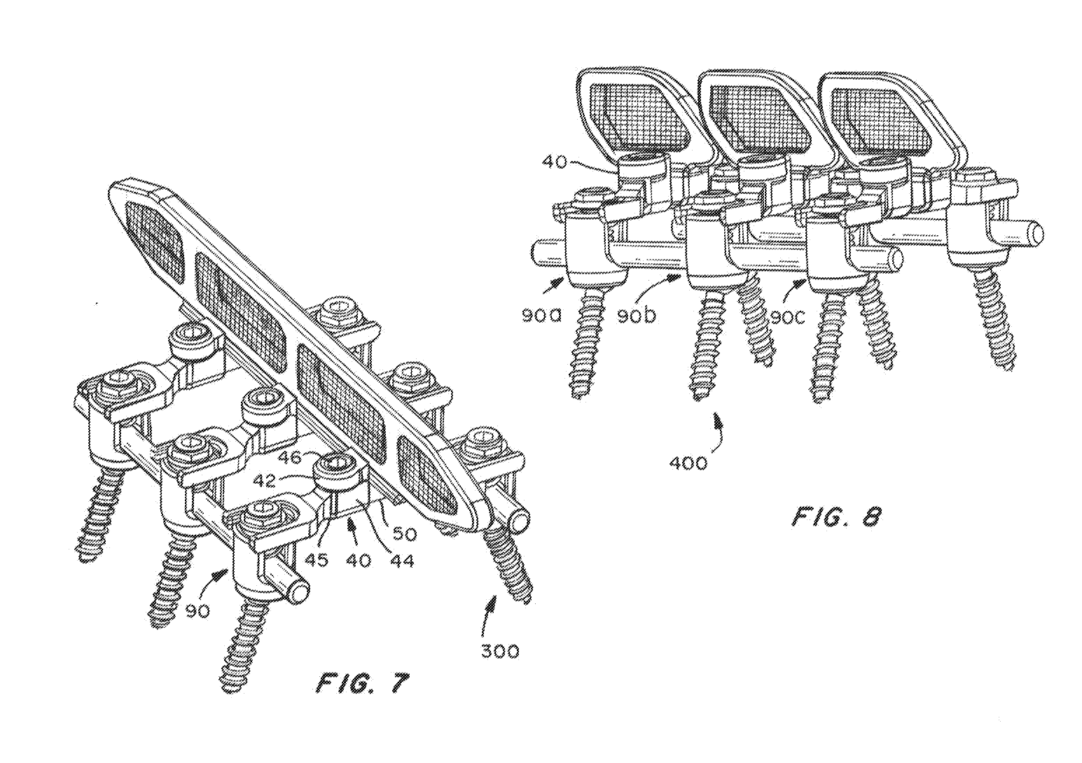

FIGS. 6A-E, 7, 8, and 9A illustrate exemplary fixation devices that attach to one or more cross connectors, where the cross connectors contain a pair of connection portions (72a and 72b) that attach to the screw heads (90a, b, and c) of the bone screws. The connection portions (72a and 72b) contain a U-shaped end having a suitable size and shape to attach to the screw head (90). The interior portion of the U-shaped end contains a flange (74). When the connection portion is attached to the screw head, the interior flange (74) is placed on the top surface (92) of a rod receiving portion (91) which connects the rod with the bone screw. A screw (94), optionally with a cap or washer (96) is placed on top of the flange and turned in place. Preferably the top portion of the inner surface of the rod receiving portion (91) is threaded to receive the screw. As the screw is turned, it pushes the rod (80) against the bottom portion of the rod receiving portion (91) until it securely connects the cross connector to the rod, which is attached to the bone screw.

### II. Methods of Using the Device

The lamina and spinous processes are removed from the vertebrae in the area of spine in which a laminectomy and/or foraminotomy (decompression of the spine) is performed. Attachments to the fascia and muscular attachments of the paraspinal musculature to the spinous process and lamina are also removed.

Paraspinal muscles refer collectively to the band of muscles next to the spine. For example, in the thoracic spine, the paraspinal muscles consist of five sets of muscles: longissimus thoracis muscles, which connect the transverse processes of adjacent vertebrae and help extend and laterally flex the vertebral column and help rotate the ribs; iliocostalis thoracis, which are small muscles that connect the ribs; spinalis thoracis, which connect the spinous processes of adjacent vertebrae and help extend the vertebral column; spinalis thoracis connect the spinous process of one vertebra to the transverse processes of an adjacent vertebra and extend and rotate the vertebral column; and rotatores thoracis which connect the spinous process of one vertebra to the transverse process of an adjacent vertebra (short rotatores) or the vertebra two away (long rotatores) and extend and rotate the vertebral column. The lumbar paraspinal muscles are a set of three muscle groups: multifidus muscle, longissimus muscle and iliocostalis muscle fascicles.

After the surgeon places the lateral mass or pedicle screws and rods to stabilizes the patient's spine, optionally following the placement of any cross connectors to further stabilize the patient's spine, the surgeon attaches one or more fixation device(s) described herein.

For surgery in which a relatively small muscle attachment portion is needed, then the muscle attachment portion may be preassembled to the cross connector prior to insertion into the patient.

For surgery, in which longer muscle attachment portions are required, then the fixation device is attached via its connectors to the cross connectors in situ, after the cross connectors are attached to the rods or pedicle screws.

The surgeon then sutures the paraspinal muscles and fascia that were previously separated from the spine to the muscle attachment portion by attaching the sutures through the muscle attachment area, particularly along the length of the muscle attachment area.

Then the surgeon closes the wound by sewing the two split sides to each other.

The device provides a substrate for attachment of the paraspinal musculature and fascia, thereby reestablishing the natural interaction between the spine and the paraspinal muscles following a laminectomy and providing support in this portion of the spine.

The use of the fixation device following surgery not only provides greater support to the spine, it also prevents or reduces the likelihood that the paraspinal musculature will attach to the dura mater, spinal cord and/or nerves. It also reduces the likelihood of a tissue reaction, such as the formation of scar tissue and/or adhesions.

### III. Kits Containing the Device

The fixation device may be provided as part of a kit for a spinal fixation system. Optionally the kit contains a plurality of lateral mass screws or pedicle screws, at least two rods and optionally more than two rods, at least one fixation device. If the fixation device is formed only of one or more muscle attachment portions and one or more connectors, then the kit will also typically contain one or more cross connectors. The kit also contains instructions for care and insertion of the spinal fixation system.

The kit may also include tool(s) for placement of the screws, rods, cross connectors and fixation device, such as drills, taps and drivers.

Those skilled in the art will recognize, or be able to ascertain using no more than routine experimentation, many equivalents to the specific embodiments of the invention described herein. Such equivalents are intended to be encompassed by the following claims.

I claim:

1. A device for attaching paraspinal muscles or fascia following spine surgery,
wherein the device comprises a muscle attachment portion, one or more connectors, and one or more cross connectors,
wherein the muscle attachment portion contains a muscle attachment area suitable for the attachment of paraspinal muscles or fascia,
wherein the one or more cross connectors comprise an upper portion and a lower portion,
wherein the muscle attachment portion is superior to the connectors and located at approximately a 90° angle relative to the upper portion of the connectors, and
wherein the cross connectors comprise a pair of connection portions, wherein each connection portion has a configuration suitable for attachment to a fixation rod or a screw heads of a bone screw.

2. The device of claim 1, wherein the muscle attachment portion and connectors are formed from the same or different biocompatible materials.

3. The device of claim 2, wherein the biocompatible materials are not biodegradable.

4. The device of claim 2, wherein the muscle attachment portion is formed from a biodegradable material.

5. The device of claim 1, wherein the cross connector is an adjustable or a fixed cross connector.

6. The device of claim 1, wherein the muscle attachment portion further comprises one or more therapeutic or diagnostic agents.

7. The device of claim 1, wherein the muscle attachment area comprises a plurality of holes.

8. The device of claim 1, wherein the muscle attachment area comprises a material suitable for suturing.

9. The device of claim 1, wherein the one or more cross connectors are integral with the muscle attachment portion.

10. The device of claim 1, wherein the muscle attachment portion further comprises a flange at its lower region.

11. The device of claim 10, comprising a pair of cross connectors, where the first connector is located on one side of the muscle attachment portion and the second connector is located on the opposite side of the muscle attachment portion, and
wherein each connector further comprises a groove on the side proximal to the muscle attachment portion, wherein the flange fits in the groove.

12. A method for stabilizing a patient's spine following spine surgery including the step of removing the spinous process from one or more vertebrae, comprising
(a) attaching the device of claim 1 to hardware implanted in the spine, wherein the hardware is selected from the group consisting of screw heads and rods, and
(b) subsequent to step (a), attaching to the muscle attachment area in the muscle attachment portion the paraspinal muscles and fascia that previously attached to the spinous process.

13. The method of claim 12, wherein the paraspinal muscles and fascia are attached via sutures.

14. The method of claim 12, wherein step (a) comprises attaching the connection portion of each of the cross connectors to a screw head.

15. The method of claim 12, wherein step (a) comprises attaching the connection portion of each of the cross connectors to a rod.

16. The method of claim 12, wherein the muscle attachment portion of the device comprises one or more analgesics or anesthetics, and wherein the muscle attachment portion releases an effective amount of the one or more analgesics or anesthetics to reduce pain in the spine for a period of time ranging from one week to 8 weeks following implantation.

17. The method of claim 12, wherein the muscle attachment portion of the device comprises one or more drugs to reduce scar and adhesion formation, and wherein the muscle attachment portion releases an effective amount of the drugs to reduce scar or adhesion formation in the spine for a period of time ranging from one week to 8 weeks following implantation.

18. The method of claim 12, wherein step (b) is effective to reduce the attachment of the paraspinal musculature to the dura mater, spinal cord, nerves, or a combination thereof.

19. A kit for spinal surgery comprising two or more pedicle or lateral screws, two or more rods, two or more screw heads, and the device of claim 1.

20. The kit of claim 19, comprising two or more cross connectors.

21. The kit of claim 19, comprising two or more segmented muscle attachment portions.
